(19) United States
(12) Patent Application Publication
(10) Pub. No.: US 2011/0313479 A1
(43) Pub. Date: Dec. 22, 2011

(54) SYSTEM AND METHOD FOR HUMAN ANATOMIC MAPPING AND POSITIONING AND THERAPY TARGETING

(76) Inventor: Philip Rubin, Rochester, NY (US)

(21) Appl. No.: 12/820,598

(22) Filed: Jun. 22, 2010

(51) Int. Cl. A61B 6/00; G06K 9/00

(52) U.S. Cl. 607/1; 382/131

(57) ABSTRACT

A method of image processing, comprising obtaining a three-dimensional standard animal body image having three intersecting two-dimensional planes and comprising an anatomical landmark; obtaining a three-dimensional radiographic image of a patient animal having a corresponding anatomical landmark; and comparing the standard animal body image with the radiographic image by: identifying the location of the anatomical landmark on one two-dimensional plane in the standard animal body image; automatically propagating the identified location of the anatomical landmark to the other two two-dimensional planes in the standard animal body image; identifying the location of the anatomical landmark in the radiographic image of the patient animal; and morphing the radiographic image of the patient animal to the standard animal body image by deforming the radiographic image of the patient animal to cause the locations of the landmark on the radiographic image and the standard animal body image to overlap.

### FIELD

The invention relates to computer aided diagnosis and therapy planning, in particular, computer aided diagnosis and therapy planning using a standard animal body image and radiographic imagery.

### BACKGROUND

The increasing importance of cross-sectional image as the single most important clinical approach for viewing a patient's anatomy, both in primary care as well as the specialty medicine, has rendered the familiarity with sectional anatomy highly desirable in handing three-dimensional radiographic images. There is a need in the art to blend a standard sectional anatomy with radiographic images to give clinicians better tools for interpretation and diagnosis.

### SUMMARY

Some embodiments of the current invention may provide a method for processing radiographic images, comprising: obtaining a three-dimensional standard animal body image having three intersecting two-dimensional planes and comprising one anatomical landmark including an anatomical feature identifiable in all bodies of the animal; obtaining a three-dimensional radiographic image of a patient animal; and comparing the standard animal body image with the radiographic image by manually identifying the location of the anatomical landmark on one two-dimensional plane in the three-dimensional standard animal body image; automatically propagating the identified location to the other two two-dimensional planes in the three-dimensional standard animal body image; identifying the location of the anatomical landmark in the three-dimensional radiographic image; and morphing the three-dimensional radiographic image to the standard animal body image by deforming the three-dimensional radiographic image to cause the locations of the landmark on the three-dimensional radiographic image and the standard animal body image to overlap.

Some embodiments of the current invention provide a method for processing radiographic images, comprising: obtaining a three-dimensional standard animal body image having three intersecting two-dimensional planes, wherein said three-dimensional standard animal body image includes a vasculature tree; obtaining a three-dimensional patient map; comparing the standard animal body image and the patient map by identifying the locations of the anatomical landmark in the standard animal body image and the three-dimensional radiographic image; morphing the three-dimensional radiographic image to the standard animal body image by deforming the three-dimensional radiographic image to cause the locations of the landmark on the three-dimensional radiographic image and the standard animal body image to overlap; fusing the three dimensional radiographic image and the standard animal body image to produce a three dimensional representation of the identified anatomical landmark; and visualizing the vasculature tree relative to the corresponding location of the identified anatomical landmark on the produced three-dimensional representation.

Some embodiments of the current invention provide a system for viewing multi-dimensional images of an animal body, comprising a computer system comprising a storage device to receive a three-dimensional radiographic image of a patient body and a three-dimensional standard animal body image, wherein the three-dimensional radiographic image corresponds to each plane of the three-planar view of the animal body, and the three-dimensional standard animal body image comprises a vasculature tree; means for identifying an anatomical landmark in both the three-dimensional standard image body and the three-dimensional radiographic image; means for identifying the locations of the anatomical landmark in the standard animal body image and the three-dimensional radiographic image; means for morphing the three-dimensional radiographic image to the standard animal body image by deforming the three-dimensional radiographic image to cause the locations of the landmark on the three-dimensional radiographic image and the standard animal body image to overlap; means for fusing the three dimensional radiographic image and the standard animal body image to produce a three dimensional representation of the identified anatomical landmark; and a display device to visualize the vasculature tree relative to the corresponding location of the identified anatomical landmark on the produced three-dimensional representation.

### BRIEF DESCRIPTION OF THE DRAWINGS

The foregoing and other features of the invention will be apparent from the following, more particular description of exemplary embodiments of the invention, as illustrated in the accompanying drawings wherein like reference numbers generally indicate identical, functionally similar, and/or structurally similar elements. The left most digits in the corresponding reference number indicate the drawing in which an element first appears.

FIG. 1 shows a flow chart of a method for processing radiographic images according to some embodiments of the current invention.

FIG. 2 shows another flow chart of a method for guiding radiation treatment according to some embodiments of the current invention.

FIG. 3A shows a coronal view of a standard image of a human head and neck with a vasculature tree and surrounding lymph nodes according to some embodiments of the current invention.

FIG. 3B shows a sagittal view of the standard image of a human head and neck with the vasculature tree and surrounding lymph nodes according to some embodiments of the current invention.

FIG. 4 shows an axial view of the standard image of a human head and neck with the vasculature tree and surrounding lymph nodes as well as a morphed computed tomography image with the lymph nodes fused according to some embodiments of the current invention.

FIG. 5 shows an axial view of a standard image of a human thorax with lymph nodes as well as a morphed computed tomography image with the lymph nodes fused according to some embodiments of the current invention.

FIG. 6A shows a coronal view of a standard image of a human head and neck as well as a morphed computed tomography image with color codings showing tolerance to radiation according to some embodiments of the current invention.

FIG. 6B shows a sagittal view of a standard image of a human head and neck as well as a morphed computed tomography image with color codings showing tolerance to radiation according to some embodiments of the current invention.

FIG. 7 shows a coronal, sagittal, and axial view of a fused image of a human head and neck with color codings showing staging of a cancerous condition according to some embodiments of the current invention.

FIG. 8 shows a system for viewing multi-dimensional images of an animal body according to some embodiments of the current invention.

### DEFINITIONS

In describing the invention, the following definitions are applicable throughout (including above).

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a personal digital assistant (PDA); a portable telephone; application-specific hardware to emulate a computer and/or software, such as, for example, a digital signal processor (DSP), a field-programmable gate array (FPGA), a chip, chips, or a chip set; an optical computer; a quantum computer; a biological computer; and an apparatus that may accept data, may process data in accordance with one or more stored software programs, may generate results, and typically may include input, output, storage, arithmetic, logic, and control units.

“Software” may refer to prescribed rules to operate a computer or a portion of a computer. Examples of software may include: code segments; instructions; applets; pre-compiled code; compiled code; interpreted code; computer programs; and programmed logic.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a memory chip; and/or other types of media that can store machine-readable instructions thereon.

A “computer system” may refer to a system having one or more computers, where each computer may include a computer-readable medium embodying software to operate the computer. Examples of a computer system may include: a distributed computer system for processing information via computer systems linked by a network; two or more computer systems connected together via a network for transmitting and/or receiving information between the computer systems; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet. Exemplary networks may operate with any of a number of protocols, such as Internet protocol (IP), asynchronous transfer mode (ATM), and/or synchronous optical network (SONET), user datagram protocol (UDP), IEEE 802.x, etc.

A “real-time” process may refer to a process performed on a computer or computer system that controls an on-going process and delivers its outputs (or controls its inputs) not later than the time when these are needed for effective control. A “real-time” image may refer to a still image or a moving image, typically useful in X-ray, CT, or MR imaging.

Moreover, as used herein, “three-dimensional” may refer to spatial dimensions, while embodiments of the invention may incorporate multi-dimensional characteristics through the addition of other dimensions (e.g., temporal) to reflect changes in time, etc.

### DETAILED DESCRIPTION OF EMBODIMENTS OF THE PRESENT INVENTION

Exemplary embodiments of the invention are discussed in detail below. While specific exemplary embodiments are discussed, it should be understood that this is done for illustration purposes only. In describing and illustrating the exemplary embodiments, specific terminology is employed for the sake of clarity. However, the invention is not intended to be limited to the specific terminology so selected. A person skilled in the relevant art will recognize that other components and configurations may be used without parting from the spirit and scope of the invention. It is to be understood that each specific element includes all technical equivalents that operate in a similar manner to accomplish a similar purpose. Each reference cited herein is incorporated by reference. The examples and embodiments described herein are non-limiting examples.

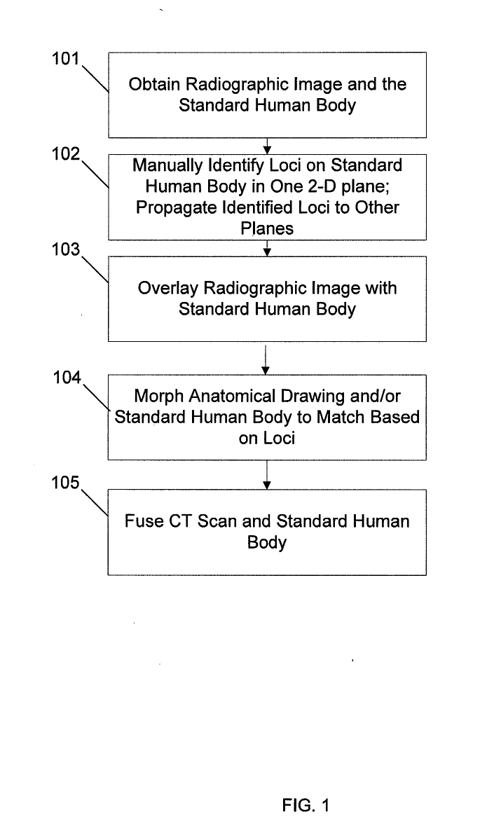

FIG. 1 is a flowchart for fusing and morphing a radiographic image of a human body to a standard human body according to some embodiments of the current invention. In block 101, a radiographic image of a human patient may be obtained along with the standard human body. The radiographic image may be, for example, a three-dimensional radiology scan for the patient under observation. The radiology scan may be, for example, X-ray, computed tomography (CT), magnetic resonance imaging (MRI), positron emission tomography (PET), single positron emission computed tomography (SPECT), or variations thereof. The radiographic image may be a three-dimensional image of the human patient.

In block 102, the anatomical landmarks, or loci, that may be present in the radiographic image may be identified in the standard human body and the radiographic image. The Human Anatomic Mapping and Positioning System (HUMAPS) may include a standard human body image as a three-dimensional map having three intersecting orthogonal planes. HUMAPS may also include three-dimensional coordinates for specified anatomic landmarks within the standard animal body image. For example, a number of anatomic landmarks may be defined within a standard human body image. For example, there may be twenty-nine defined anatomic landmarks, or loci, based on the critical anatomical structures located at those loci. The twenty-nine loci may be correlated to accepted surface anatomical features used in physical diagnosis, and may be imaged from cephalad to caudad in transverse sections. Loci may be located in rigid structures, for example, in bones, or they may be located in non-rigid structures, for example soft tissue. In particular, the landmarks may be manually identified in one plane and then automatically propagated to other planes, for example, by a programmed computer. Landmarks in the radiographic image may be identified in a similar fashion. Other relevant anatomical features, locations, and landmarks, such as, for example, organs, tissues, vasculature, and tumors, may also be identified in the radiographic image. The loci may already have been assigned three-dimensional coordinates, or human anatomic mapping and positioning system (HUMAPS) zipcodes, based on their position in the standard human body. The HUMAPS zipcodes have been described in published PCT Application No. WO 2007/117695 A2, incorporated by reference.

In block 103, the radiographic image may be overlaid with the correlated standard human body. The correlated standard human body may use three-dimensional coordinates or HUMAPS zipcodes assigned to the loci present in the radiographic image. The correlated standard human body may also use the information related to the acquisition of the radiographic image. The correlated standard human body may be overlaid on the radiographic image semi-transparently, so that both the anatomical drawing of the standard human body and the radiographic image are visible at the same time. Visible leader lines and labels in the radiographic image may be transferred directly to the standard human.

In block 104, the radiographic image and overlaid correlated anatomical drawing may be morphed so that the loci common to both images overlap, resulting in congruency between the images. The morphing may involve image deformation such as horizontal stretching, vertical stretching, magnification, or any other image manipulation. The morphing may be based on the calculatable correlation between the anatomic landmarks locations in the standard human body, using the loci identified in the radiographic image. The morphing may make use of non-linear image registration, based on non-linear or deformable matrix transformation. Triangulation may be used to establish relationships between the identified loci in order to facilitate the morphing process. Software tools, such as, for example, Automatic Image Registration or Morpheus Photo Morpher v3.01 (available from Morpheus Software, LLC of Santa Barbara, Calif., USA) may be employed to accomplish morphing. Morphing may be performed on one image, morphing the image so its loci match up with the loci of the non-morphed image, or morphing may be performed on both images at the same time, deforming each image until the loci present in both images match up. The leader lines and labels in the standard human body may be transferred to the radiographic image after morphing.

In block 105, the morphed images may be fused into a single image. The morphed radiographic image and standard human body may be fused together to create a single, composite image containing the information present in both images. This may include location markings, for example, the location of a tumor on the radiographic image, and information such as the coloration, leader lines and labels from the anatomical drawing of the standard human body. The fused image may be presented to a viewer electronically, or as a printout. An electronic fused image may have an option allowing for a viewer to switch between viewing the radiographic image or the anatomical drawing individually and viewing the fused image. The opaqueness of each component image of the fused image may be adjusted to vary the blending. For example, that anatomical drawing may be made to be 100% opaque, while the CT-scan may be made to be 50% opaque, allowing for the anatomical drawing to be viewed through the CT-scan in the fused image. The coloration and color saturation of each image may also be adjusted. For example, that coloration of the anatomical drawing may be switched on and off, between colored and gray-scale version. Color saturation of the coloration of an image may also be adjusted gradually, for example, starting at 0% color saturation, or gray-scale, and proceeding to 100% color saturation in increments, for example, 1% increments. Coloration, leader lines and labels present on the standard human body may be preserved, or they may be removed, depending on the preference of the fused image creator or viewer.

In another embodiment, the leader lines and labels in one of the radiographic image or the standard human body may be transferred and preserved in the fused image by morphing the component images. The relative position of a leader line may be indicated on images of the underlying internal anatomy to facilitate identification or the internal anatomy.

In another embodiment, three-dimensional fused image may be color coded to guide treatment of the patient. The color coding may represent, for example, radiation tolerance level. The color coding may be used, for example, by radiation, medical, and surgical oncologists in the treatment of their patients. The color coded locational information may be used for the targeting the treatment to the locations.

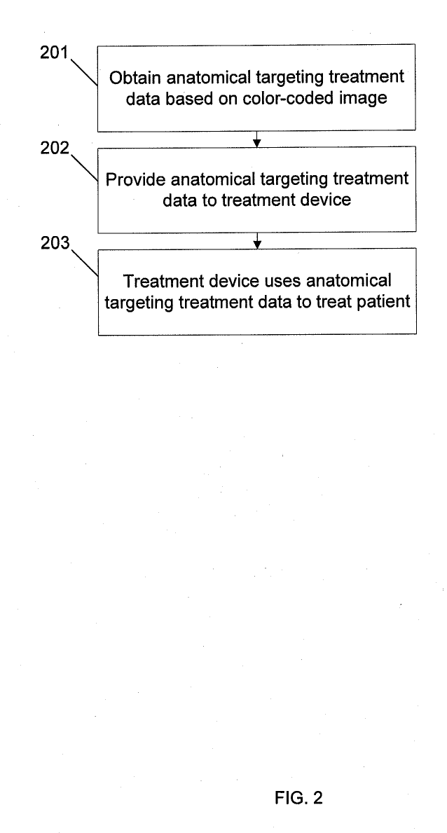

FIG. 2 is a flowchart for using the color coded fused image to guide treatment a patient according to some embodiments of the current invention. In block 201, the color coded fused image is obtained for a patient. The location for treatment may be contained in the fused images using the three-dimensional grid applied to the standard human body. In one embodiment, correlated sets of anatomical drawings and radiographic images according to three-planar anatomy may be provided as a compilation. The correlated sets may additionally include fused images. The correlated sets may be provided, for example, in book form, in e-book form, software form, or as a website or other internet accessible data service, or in any other suitable form. The correlated sets may contain three-planar images covering an entire standard animal body image, or a specific region of an animal body, and may be indexed and searchable by region, by names given to anatomic locations and anatomic landmarks, or by three-dimensional coordinates. For example, a software program may accept as input three-dimensional coordinates and provide in response the correlated sets of three-planar images containing those coordinates.

In block 202, the anatomical targeting treatment data contained in the color coded fused image may be provided to the treatment device. The treatment device may be any medical device used to treat a patient, including, for example, external radiation therapy systems using high energy X-day, α ray, β ray, γ ray, radioisotope radiation system, microwave system, high intensity ultrasound system, etc. The anatomical targeting treatment data may be transferred to the treatment device electronically, for example over a wired or wireless network, through the use of a removable computer readable medium such as a CD, DVD, floppy disk, or flash memory device, or it may be manually input into the treatment device. The treatment device may receive other treatment parameters along with the anatomical targeting treatment data. For example, the treatment device may receive the treatment dosage, patient height and weight, among other parameters.

In block 203, the treatment device may use the anatomical targeting data to treat the patient. The treatment device may provide treatment to the location within the patient's body corresponding to the anatomical targeting treatment data. The treatment device may translate the anatomical targeting data based on patient height and weight, by, for example, performing a translation from the standard animal body image to the patient's body using the loci in the treatment area. This may be done to translate the three-dimensional coordinates, for example, the HUMAPS zipcode, into the correct physical location on the patient.

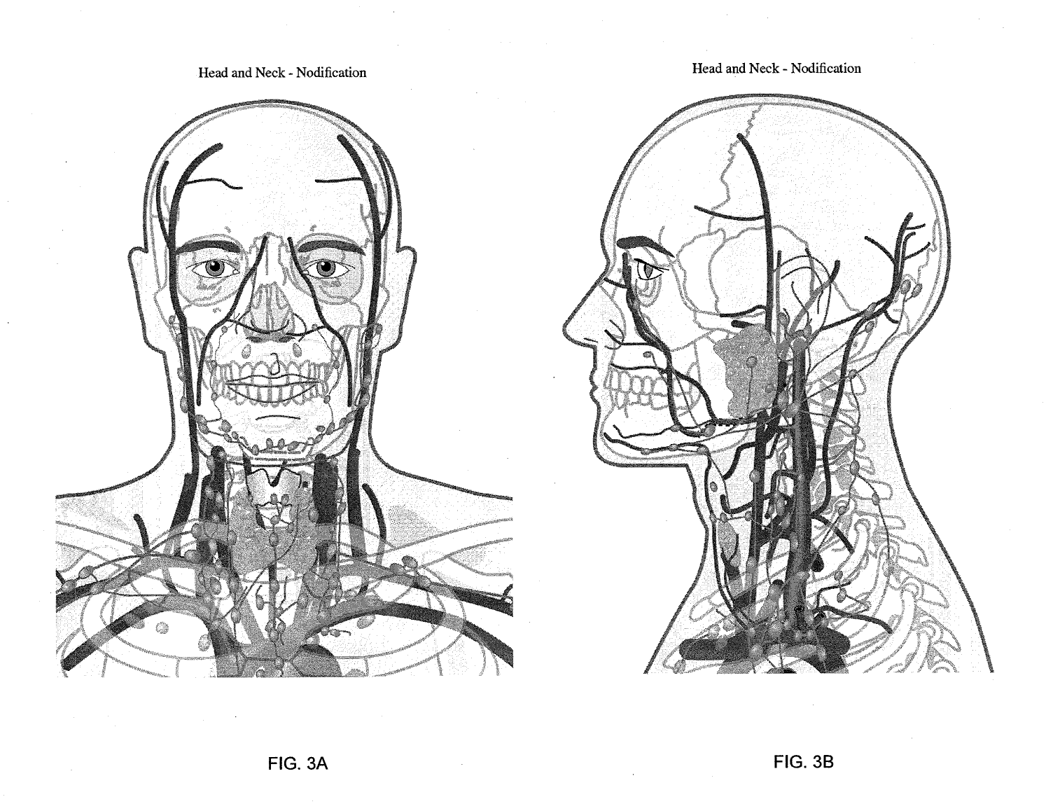

FIG. 3A shows a coronal view of a standard image of a human head and neck with a vasculature tree and surrounding lymph nodes according to some embodiments of the current invention.

FIG. 3B shows a sagittal view of the standard image of a human head and neck with the vasculature tree and surrounding lymph nodes according to some embodiments of the current invention.

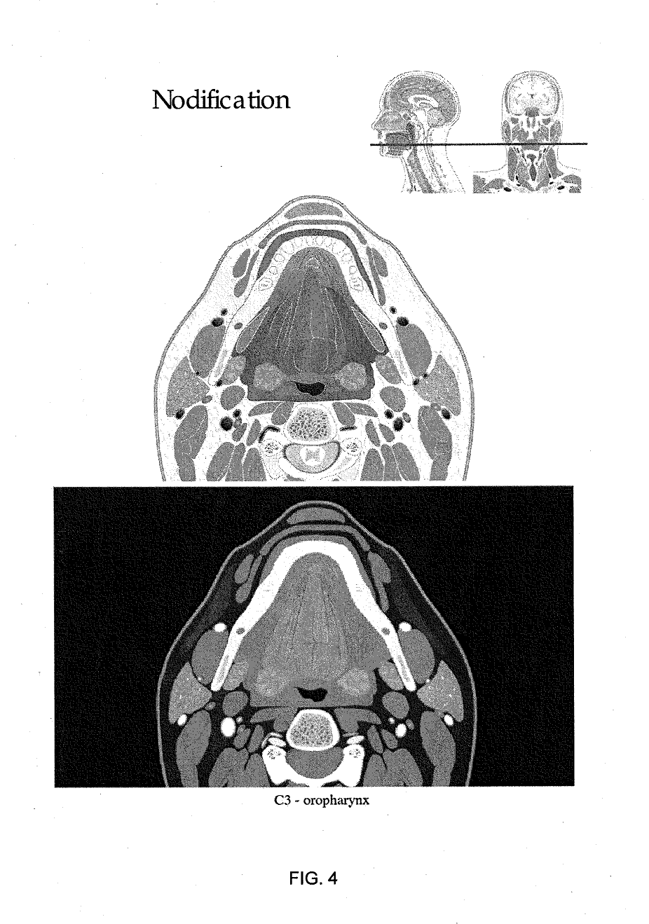

FIG. 4 shows an axial view of the standard image of a human head and neck with the vasculature tree and surrounding lymph nodes as well as a morphed computed tomography (CT) image with the lymph nodes fused according to some embodiments of the current invention. The top row shows the relative location of the axial slice along the head-foot direction of a standard human body. The central row shows an axial view of a standard human body and the bottom row shows the axial view of the fused image with lymph nodes overlaid. The fused image enables an oncologist to visualize the locations of the vasculature tree relative to, for example, a cancerous organ. The relative location enables the oncologist to differentiate vessels entering into the cancerous organ from those exiting from the cancerous organ. For example, portions of said vasculature tree feeding into the location of the anatomical landmark of the cancerous organ in the radiographic image of the patient animal may be identified. Based on the identified portions of vasculature feeding into the cancerous organ, a quantity corresponding to the blood input characteristic of the cancerous organ may be obtained. In addition, the location of the lymph nodes relative to the cancerous organ enables the oncologist to grade the cancerous organ, for example, according to a metastasis potential. The CT image itself may also reveal if the lymph nodes are cancerous.

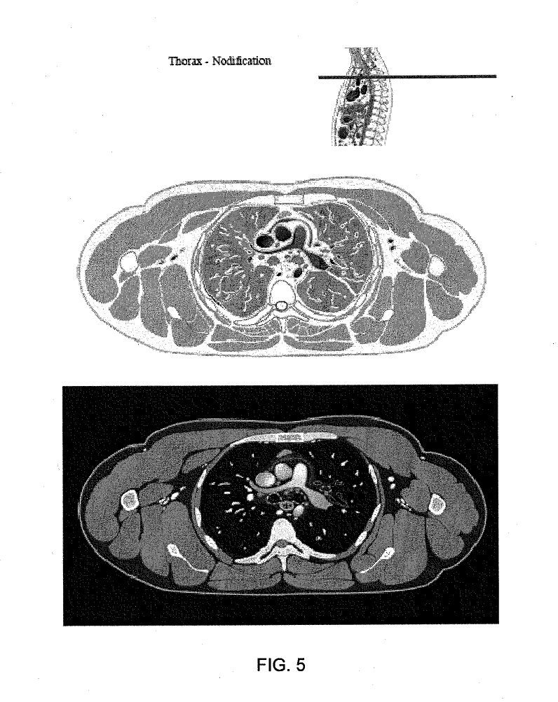

FIG. 5 shows an axial view of a standard image of a human thorax with lymph nodes as well as a morphed computed tomography image with the lymph nodes fused. The top row shows the relative location of the axial slice along the head-foot direction of a standard human body. The central row shows an axial view of a standard human body and the bottom row shows the axial view of the fused image with lymph nodes overlaid. The location of the lymph nodes relative to the cancerous organ enables the oncologist to grade the cancerous organ, for example, according to a metastasis potential. The CT image itself may also reveal if the lymph nodes are cancerous.

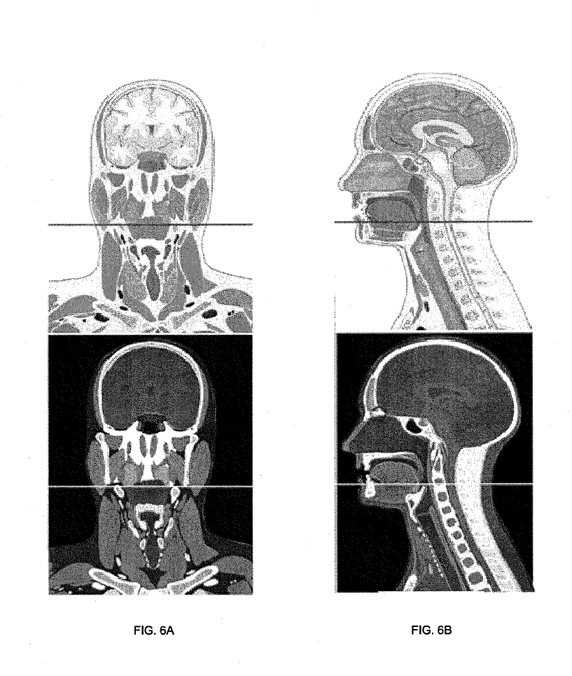

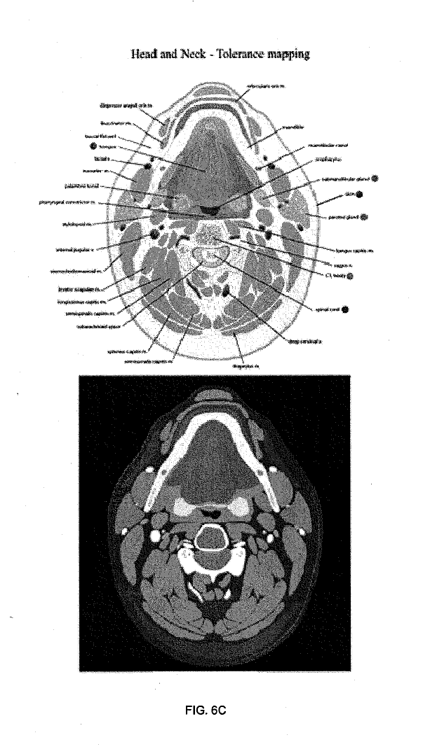

FIG. 6A-C shows a coronal, sagittal, and axial view of a standard image of a human head and neck as well as a morphed computed tomography image with color codings showing tolerance to radiation according to some embodiments of the current invention. The upper row shows an anatomical drawing from the standard human body. The lower row shows the fused image with color coding indicating tolerance to radiation dosage. Here, maroon means most resistant. For example, the skin and spinal cord are coded maroon because of their resistance. Pink means second most resistant. For example, the parotid gland and submandibular gland are pink coded. Brown means third most resistant. For example, the tongue is brown coded. Yellow means least resistant. For example, the palantine tonsil is yellow coded. Further, all the veins are blue coded. The color coding system affords a oncologist the ability to target radiation therapy to the organs according to their resistance to radiation therapy as described in association with FIG. 2.

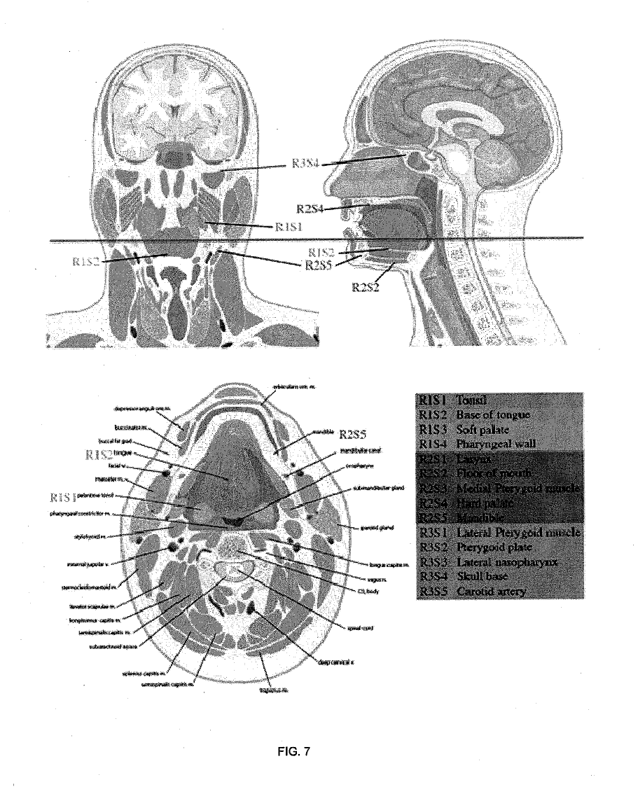

FIG. 7 shows a coronal, sagittal, and axial view of a fused image of a human head and neck with color codings showing staging of a cancerous condition on an oncology index according to some embodiments of the current invention. For example, on the color scale, the tonsil is graded as most cancerous, followed by base of the tongue. Soft palate and pharyngeal wall are graded next in pink. The larynx, floor of mouth, medial pterygoid muscle, hard palate, and mandible are coded red and are less cancerous than those in pink. The lateral pterygoid muscle, pterygoid plate, lateral nasopharynx, skull base, and the carotid artery are not cancerous and coded in gray.

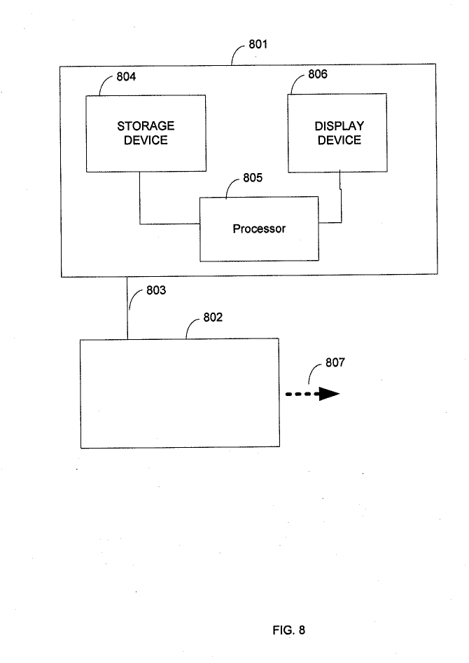

FIG. 8 shows a system for viewing multi-dimensional images of an animal body according to some embodiments of the current invention. The system may comprise a computer system 801 and a radiation delivery system 802, in communication with each other via link 803. Link 803 may be may be wired or wireless. A wired link may be, for example, a serial cable, a parallel cable, an Ethernet cable, a USB cable, a firewire cable, a fiber-optic cable, etc. A wireless link may be, for example, a radio-frequency (RF) link based on the Bluetooth or IEEE 802.11 protocols, an infrared link based on the infrared data association (IrDA) specifications. Links 803 is not limited to the above particular examples and can include other existing or future developed communications link without departing from the current invention.

Computer system 802 comprises a storage device 804, a display device 805, and a processor 806. Storage device 804 may receive a three-dimensional radiographic image of a patient body and a three-dimensional standard animal body image. The radiographic image may corresponding to each plane of the three-planar view of the animal body. The standard animal body image may comprise a vasculature tree and a three-dimensional radiographic images of the animal body.

Processor 805 may be in communication with storage device 804 to receive and execute instructions for identifying an anatomical landmark in both the three-dimensional standard image body and the three-dimensional radiographic image. Processor 805 may further receive and execute instructions for identifying the locations of the anatomical landmark in the standard animal body image and the three-dimensional radiographic image. Processor 805 may also receive and execute instructions for morphing the three-dimensional radiographic image to the standard animal body image by deforming the three-dimensional radiographic image to cause the locations of the landmark on the three-dimensional radiographic image and the standard animal body image to overlap. Processor 805 may additionally receive and execute instructions for fusing the three dimensional radiographic image and the standard animal body image to produce a three dimensional representation of the identified anatomical landmark.

Display device 806 may be in communication with processor 805 to receive the three dimensional radiographic image fused with the standard animal body image. Display device 806 may visualize the vasculature tree relative to the corresponding location of the identified anatomical landmark on the fused three-dimensional representation. Display device 806 may be, for example, a cathode ray tube (CRT) monitor, a liquid crystal display (LCD) monitor, a digital light projection (DLP) monitor, a projector display, a laser projector, a plasma screen, an organic light emitting diode (OLED), etc. However, display device 101 is not limited to these particular examples. It can include other existing or future developed display devices without departing from the scope of the current invention.

Radiation delivery system 802 may be in communication with computer system 801 to receive information to receive treatment information corresponding to the radiographic image fused with the standard animal body image. The radiation energy may be one of a X-ray energy, a α-ray energy, β-ray energy, a γ-ray energy, a microwave energy, an ultrasound energy, or combinations thereof.

In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A method, comprising:
obtaining a three-dimensional standard animal body image having three intersecting two-dimensional planes and comprising an anatomical landmark;
obtaining a three-dimensional radiographic image of a patient animal having a corresponding anatomical landmark; and
comparing the standard animal body image with the radiographic image by:
identifying the location of the anatomical landmark on one two-dimensional plane in the standard animal body image;
automatically propagating the identified location of the anatomical landmark to the other two two-dimensional planes in the standard animal body image;
identifying the location of the anatomical landmark in the radiographic image of the patient animal; and
morphing the radiographic image of the patient animal to the standard animal body image by deforming the radiographic image of the patient animal to cause the locations of the landmark on the radiographic image and the standard animal body image to overlap.

2. The method of claim 1, wherein said anatomical landmark is an organ that is cancerous in the patient animal.

3. The method of claim 1, wherein said anatomical landmark is an organ that has been subjected to radiation therapy.

4. The method of claim 1, wherein said standard animal body image includes a vasculature tree.

5. The method of claim 4, further comprising:
identifying, in the radiographic image of the patient animal, portions of said vasculature tree feeding into the location of the anatomical landmark.

6. The method of claim 4, further comprising:
quantifying a blood input characteristic of the anatomical landmark based on the identified portions of said vascular tree.

7. The method of claim 4, wherein the vasculature tree further comprise a plurality of lymph nodes.

8. The method of claim 7, further comprising:
classifying the radiographic image on an oncology index according to the location of the plurality of lymph nodes relative to the anatomical landmark.

9. The method of claim 7, further comprising:
determining if at least one of the plurality of lymph nodes is cancerous.

10. The method of claim 1, further comprising:
color coding the morphed radiographic image, wherein said color coding corresponds to one of a desired radiation dose for treating the anatomical landmark, a tolerance level to an ionizing radiation, or a metastasis index.

11. The method of claim 10, further comprising:
planning a radiation treatment based on the color coding.

12. The method of claim 11, wherein said treatment is one of an external beam radiation, a radioisotope therapy, an ultrasound therapy, a microwave therapy, or combinations thereof.

13. The method of claim 12, further comprising:
prognosing a medical condition based on the color coding.

14. A computer-readable medium, containing software, which software, when executed by a computer, causes the computer to execute the method of claim 1.

15. A method for processing radiographic images, comprising:
obtaining a three-dimensional standard animal body image having three intersecting two-dimensional planes, wherein said three-dimensional standard animal body image includes a vasculature tree;
obtaining a three-dimensional radiographic image of a patient body;
comparing the standard animal body image and the radiographic image of the patient body by:
identifying the location of an anatomical landmark in the standard animal body image and the radiographic image of the patient body;
morphing the radiographic image of the patient body to the standard animal body image by deforming the radiographic image to cause the locations of the anatomical landmark on the radiographic image of the patient body and the standard animal body image to overlap;
fusing the radiographic image of the patient body and the standard animal body image to produce a three dimensional representation of the identified anatomical landmark; and
visualizing the vasculature tree relative to the corresponding location of the identified anatomical landmark on the fused image.

16. The method of claim 15, wherein the vasculature tree further comprises a plurality of lymph nodes.

17. The method of claim 16, further comprising:
classifying the fused image on an oncology index according to the location of the plurality of lymph nodes relative to the anatomical landmark.

18. The method of claim 15, further comprising:
color coding the fused image, wherein said color coding corresponds to one of a desired radiation dose for treating the medically relevant organ, a tolerance level to an ionizing radiation, or a metastasis index.

19. A computer-readable medium, containing software, which software, when executed by a computer, causes the computer to execute the method of claim 14.

20. A system for viewing multi-dimensional images of an animal body, comprising:
a computer system comprising:
a storage device having a three-dimensional radiographic image of a patient body and a standard animal body image, wherein the radiographic image of the patient body has data corresponding to each plane of the standard animal body image, and the standard animal body image comprises a vasculature tree;
a processor, in communication with the storage device, to identify an anatomical landmark in both the three-dimensional standard image body and the radiographic image of the patient body, identify the locations of the anatomical landmark in the standard animal body image and the radiographic image of the patient body, morph the radiographic image of the patient body to the standard animal body image by deforming the radiographic image to cause the locations of the landmark on the radiographic image of the patient body and the standard animal body image to overlap, and to fuse radiographic image of the patient body and the standard animal body image to produce a three dimensional representation of the identified anatomical landmark; and
a display device, in communication with the processor and the storage device, to visualize the vasculature tree relative to the corresponding location of the identified anatomical landmark on the fused image.

21. The system of claim 20, further comprising:
a radiation delivery system to deliver a radiation energy to the patient body, wherein
the therapy system is in communication with the computer system to receive treatment information corresponding to the radiographic image fused with the standard animal body image, and
the radiation energy is one of a X-ray energy, a α-ray energy, β-ray energy, a γ-ray energy, a microwave energy, an ultrasound energy, or combinations thereof.

22. The system of claim 20, wherein
the vasculature tree further comprises surrounding lymph nodes.
